(19) United States
(12) Patent Application Publication
(10) Pub. No.: US 2023/0402163 A1
(43) Pub. Date: Dec. 14, 2023

(54) PATIENT SURGERY CANCELLATION PREDICTION SYSTEM

(71) Applicant: SurgIP Inc., Oshawa (CA)

(72) Inventors: DALIA GABER MESSIHA WAHBA, Khalifa City (AE); NUR-UL-ISLAM ANJUM, Ajman (AE)

(21) Appl. No.: 18/331,895

(22) Filed: Jun. 8, 2023

Related U.S. Application Data
(60) Provisional application No. 63/350,229, filed on Jun. 8, 2022.

(51) Int. Cl. G16H 40/20; G16H 10/20; G10L 15/02; G10L 15/22; G10L 15/183

(52) U.S. Cl. CPC G16H 40/20; G16H 10/20; G10L 15/02; G10L 15/22; G10L 15/183

(57) ABSTRACT

A surgical/medical appointment schedule adherence prediction system is described that comprises a software application that provides effective and practical scheduling solutions for clinics and operating rooms by allowing responsibilities concerning medical or surgical appointment-related tasks to be tracked and check when the tasks are completed. An advance warning may be provided to the clinic or the hospital to cancel the appointment and reallocate the time to another patient if the probability of a no show is high. In providing such a proactive tool to clinics and hospitals, the preparation cost and the opportunity cost for doctors, surgeons and other medical practitioners may be reduced.

### CROSS-REFERENCE TO RELATED APPLICATION

This application claims the benefit of U.S. Provisional Patent Application No. 63/350,229 filed Jun. 8, 2022, and the entire contents of U.S. Provisional Patent Application No. 63/350,229 are hereby incorporated herein in its entirety.

### BACKGROUND

No shows for scheduled appointments in medical clinics cost the healthcare system millions of dollars. The cost of no shows is significant across the western hemisphere according to two separate studies. No shows rates are between 10% and 25% in Canada varying by region. In the case of a no-show to a clinic, two patients lose—the one who was supposed to receive care and the other who could have been scheduled for that time.

Same day cancelations of surgeries cost the hospitals millions of dollars each year. Surgeries can account for 60% of the hospitals revenue; thus, any reduction in the number of cancelations will improve the income and cost situation.

A large number of last minute surgery cancellations may be preventable, but there has not yet been a reliable system to provide this secure service. It would be beneficial for hospitals to have enough advance notice to avoid unnecessary set up cost or opportunity cost for the staff. Such a system would also enable better utilization of hospital resources for other available patients.

### SUMMARY

The following summary is provided to introduce a selection of concepts in a simplified form that are further described below in the detailed description. This summary is not intended to identify key features or essential features of the claimed subject matter, nor is it intended to be used to limit the scope of the claimed subject matter.

In accordance with the teachings herein, there is provided at least one embodiment of a system that executes a software application that provides effective and practical scheduling solutions for clinics, operating rooms and other medical facilities that schedule appointments, by allowing each party to take note of their responsibilities concerning the medical or surgical appointment-related tasks and check when the tasks are completed. The system may be configured, based on analysis that is performed, to provide an advance warning to the clinic or the hospital to cancel the appointment and reallocate the time to another patient if the probability of a no show is high. In providing such a proactive tool to clinics and hospitals, the goal is to reduce and/or prevent the preparation cost and the opportunity cost to the doctors and surgeons from being wasted.

In at least one embodiment, there is included a system that provides medical facilities, such as hospitals, with a predictive feature to identify the probability of surgery cancellations. The system utilizes a smart chatbot that can take input from patients, and analyze the behaviors of the patients based on pattern of communication with the patient to identify a trigger for an increased likelihood of a potential no show. Once such a trigger has been identified, the system can send emails, messages, and/or other forms of notifications to various stakeholders. The smart chatbot system can isolate pertinent portions of these messages, create voice transcriptions in the course of the communication and provide a data extraction feature to share pertinent data with the stakeholders.

In at least one embodiment of, the present system provides an integrated software application that connects insurance providers, hospitals, care providing teams, and patients. The software application will have a library of templates for quickly creating follow up schedules for different types of appointments, surgeries and ambulatory services. This provides a hub for each member associated with a certain surgical or medical procedure to plan and schedule events based on their needs. In at least one embodiment, a library of educational content regarding the various types of surgeries can also be provided to patients, such that the patients can make more informed decisions. In at least one embodiment, the library may be frequently updated to include most up to date information on medical services.

Upon a no show or last minute cancelation of a patient, a no show engagement trigger is generated which results in one or more corresponding messages to be sent through a communication network to one or more the stakeholders. In at least one embodiment, the no show engagement trigger may be used to update the system algorithm to provide improved performance in the prediction of behaviour for a given patient in subsequent sequences/interactions. In at least one embodiment, the system may also be configured to provide smart surveys to collect feedback from patients and improve performance.

In one aspect, in accordance with the teachings herein, there is provided at least one embodiment of a system to predict and prevent surgical cancelation. The system may comprise a plurality of user devices for patients and stakeholders to interact with each other through electronic communication, and a service management server that is in communicatively connected to the plurality of user devices, the service management server comprising a chat module, a predictive features module, and a cancelation engagement module, such that the modules work together to analyze interactions between the user devices to predict a probability of schedule adherence for medical services by the patients.

In at least one embodiment, the stakeholders may include medical service providers, insurance providers, care providers or any combination thereof.

In at least one embodiment, the user device further comprises of voice transcription and data extraction feature to extract data from communication between a patient and a stakeholder. share data with a plurality of shareholders.

In at least one embodiment, the service management server further comprises a library of templates to create follow up schedules for various types of surgeries or medical appointments.

In at least one embodiment, the user devices are further configured to enable the patients to perform electronic registrations and intake form completion.

In at least one embodiment, the user devices are configured to enable the intake form completion by voice.

In at least one embodiment, the service management server further comprises an education library.

In at least one embodiment, the user devices are configured to provide at least one smart survey to the patients.

In at least one embodiment, the predictive features module generates smart recommendations for stakeholder to reduce probability of surgery cancelation.

In another aspect, in accordance with the teachings herein, there is provided at least one embodiment of a method to predict surgery schedule cancelation. The method may comprise providing for communication between electronic devices of at least one patient and at least one stakeholder, obtaining voice transcription and data extraction from the communication, analyzing the voice transcription and data extraction to arrive at a probability of schedule adherence, and generating at least one report of the probability of schedule adherence to the at least one stakeholder.

In at least one embodiment, a no show engagement is generated upon cancelation of the at least one patient, and the no show engagement is used to assist in generating at least one report of probability of schedule adherence.

In another aspect, in accordance with the teachings herein, there is provided at least one embodiment of a non-transitory computer readable medium storing thereon program instructions, which when executed by at least one processor, configure the at least one processor for performing a method for method for predicting surgery schedule cancelation, wherein the method is defined according to any of the embodiments described herein.

It will be appreciated that the foregoing summary sets out representative aspects of embodiments to assist skilled readers in understanding the following detailed description. These and other features and advantages of the present application will be apparent from a reading of the following detailed description and a review of the appended drawings. It is to be understood that the foregoing summary, the following detailed description, the appended drawings and the specific examples, while indicated embodiments of the application, are explanatory only and given by way of illustration only and are not restrictive of various aspects claimed herein since various changes and modifications within the spirit and scope of the application will become apparent to those skilled in the art from this detailed description.

### BRIEF DESCRIPTION OF THE DRAWINGS

For a better understanding of the various embodiments described herein, and to show more clearly how these various embodiments may be carried into effect, reference will be made, by way of example, to the accompanying drawings which show at least one example embodiment, and which are now described. The drawings are not intended to limit the scope of the teachings described herein.

FIG. 1 is an example embodiment of the surgical schedule adherence prediction system comprised of user devices and a management server.

FIG. 2 is an overview of an example of a process relating to predicting surgical schedule adherence.

FIG. 3 is a flow chart showing an example embodiment of a process in accordance with the subject disclosure.

FIG. 4 is a block diagram of an example embodiment of a cloud computing system in accordance with the subject disclosure.

FIG. 5 is a block diagram of an example embodiment of a computer system in accordance with the subject disclosure.

FIG. 6 is a block diagram of an example embodiment of a mobile device in accordance with the subject disclosure.

Further aspects and features of the example embodiments described herein will appear from the following description taken together with the accompanying drawings.

### DETAILED DESCRIPTION

At least one embodiment of a system described herein included a software application that is used to provide effective and practical clinic and operating room scheduling solutions by allowing responsibilities concerning medical or surgical appointment-related tasks to be recorded and check when the actions are completed. For example, the system may be configured to provide an advance warning to the clinic or the hospital to cancel an appointment and reallocate the time to another patient if the probability of a no show is high. In providing such a proactive tool to clinics and hospitals, the goal is to reduce and/or prevent the preparation cost and the opportunity cost to the doctors and surgeons from being wasted.

In at least one embodiment, the system utilizes a number of interfaces on mobile devices and computers (e.g., laptops, desktops, tablets) to connect various patients, healthcare provides, insurance providers, and other stakeholders. The system manages the communication between the patients and the clinics/hospitals, and uses a predictive algorithm to analyze the communication. In the event of a last minute cancelation or no show, the algorithm is updated with updated no show engagement triggers so that the algorithm can make improved predictions in the future. As a result, the system constantly improves upon itself to better prepare clinics and hospitals to plan and allocate their resources.

The detailed description provided below in connection with the appended drawings is intended as a description of examples and is not intended to represent the only forms in which the present examples can be constructed or utilized. The description sets forth functions of the examples and sequences of steps for constructing and operating the examples. However, the same or equivalent functions and sequences can be accomplished by different examples.

References to “one embodiment”, “an embodiment”, “an example embodiment”, “one implementation”, “an implementation”, “one example”, “an example” and the like, indicate that the described embodiment, implementation or example can include a particular feature, structure or characteristic, but every embodiment, implementation or example may not necessarily include the particular feature, structure or characteristic. Moreover, such phrases may not necessarily refer to the same embodiment, implementation or example. Further, when a particular feature, structure or characteristic is described in connection with an embodiment, implementation or example, it is to be appreciated that such feature, structure or characteristic may be implemented in connection with other embodiments, implementations or examples whether or not explicitly described.

It should also be noted that, as used herein, the wording “and/or” is intended to represent an inclusive-or. That is, “X and/or Y” is intended to mean X or Y or both X and Y, for example. As a further example, “X, Y, and/or Z” is intended to mean X or Y or Z or any combination thereof.

Similarly, throughout this specification and the appended claims the term “communicative” as in “communicative pathway”, “communicative coupling”, and in variants such as “communicatively coupled” is generally used to refer to any engineered arrangement for transferring and/or exchanging information. Examples of communicative pathways include, but are not limited to, electrically conductive pathways (e.g., electrically conductive wires, physiological signal conduction), electromagnetically radiative pathways (e.g., radio waves, optical signals, etc.), or any combination thereof. Examples of communicative couplings include, but are not limited to, electrical couplings, magnetic couplings, radio couplings, optical couplings or any combination thereof.

A portion of the example embodiments of the systems, devices, or methods described in accordance with the teachings herein may be implemented as a combination of hardware or software. For example, a portion of the embodiments described herein may be implemented, at least in part, by using one or more computer programs, executing on one or more programmable devices comprising at least one processing element, and at least one data storage element (including volatile and/or non-volatile memory). These devices may also have at least one input device (e.g., a keyboard, a mouse, a touchscreen, and the like) and at least one output device (e.g., a display screen, a printer, a wireless radio, and the like) depending on the nature of the device.

It should also be noted that there may be some elements that are used to implement at least part of the embodiments described herein that may be implemented via software that is written in a high-level procedural language such as object-oriented programming. The program code may be written in C, C⁺⁺ or any other suitable programming language and may comprise modules or classes, as is known to those skilled in object-oriented programming. Alternatively, or in addition thereto, some of these elements implemented via software may be written in assembly language, machine language, or firmware as needed.

At least some of the software programs used to implement at least one of the embodiments described herein may be stored on a storage media or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods of the embodiments described herein may be capable of being distributed in a computer program product comprising a computer readable medium that bears computer usable instructions, such as program code, for one or more processors. The program code may be preinstalled and embedded during manufacture and/or may be later installed as an update for an already deployed computing system. The medium may be provided in various forms, including non-transitory forms such as, but not limited to, one or more diskettes, compact disks, tapes, chips, and magnetic and electronic storage. In alternative embodiments, the medium may be transitory in nature such as, but not limited to, wire-line transmissions, satellite transmissions, internet transmissions (e.g., downloads), media, digital and analog signals, and the like. The computer useable instructions may also be in various formats, including compiled and non-compiled code.

Any module, unit, component, server, computer, terminal or device described herein that executes software instructions in accordance with the teachings herein may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information, and which can be accessed by an application, module, or both. Any such computer storage media may be part of the device or accessible or connectable thereto.

Numerous specific details are set forth in order to provide a thorough understanding of one or more embodiments of the described subject matter. It is to be appreciated, however, that such embodiments can be practiced without these specific details.

Various features of the subject disclosure are now described in more detail with reference to the drawings, wherein like numerals generally refer to like or corresponding elements throughout. The drawings and detailed description are not intended to limit the claimed subject matter to the particular form described. Rather, the intention is to cover all modifications, equivalents and alternatives falling within the spirit and scope of the claimed subject matter.

In various embodiments, the present system is configured to manage a sequence of events that are generally expected to occur through a medical service schedule process. The system, in at least one example embodiment, includes software and has with interface nodes for communication to electronic devices of patients, medical service providers, insurance providers, and/or associated facilities. This software generally includes program instructions that allow each client to control the pertinent steps of managing their desired medical service when the software is executed by the system. Stakeholders in this case may be persons with interest in maintaining optimal resource allocation in the clinics or hospitals, and they may be able to monitor the interactions for better decision making in service schedule planning.

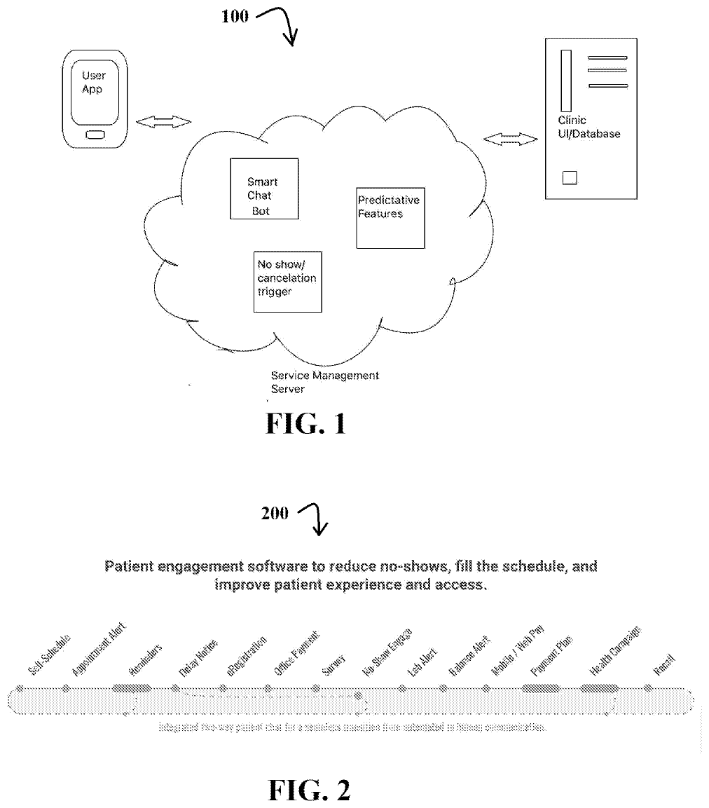

Referring to FIG. 1, an example embodiment of the system, generally designated with the numeral 100, is shown. A service management server is connected to at least one electronic device that executes a user application as well as at least one electronic device that has a clinic user interface/database. The connection may be made using known communication networks. The service management server may execute software programs that enable a number of functions including, but not limited to, a smart chat bot module, a predictive features module, and/or a cancellation engagement module for generation of no show/cancelation triggers. Each of the functions will become clear as the functions and process are explained in more detail below. The smart chat bot may be referred to as a chat module.

The triggers may include common surgery and non-invasive procedure triggers for cancellations including, but not limited to, missed lab results, lack of communication with the nurses, not providing insurance information, not providing required documentation, and/or Delay in responding to communications.

Once one or combination of some are detected, the chat bot module may create a dynamic plan of action to bring the client back to the schedule and at the same time notify the hospital and surgery team about the increased probability of cancellation. This way, a more direct and personalized communication can be initiated with the client.

For example, in at least one embodiment, a dynamic plan of action may be built around these triggers to reduce or prevent cancellations and no-shows as shown in the following examples.

A first example is Missed Lab Results. In this case once, it is determined that a patient has missed their lab results, the chat bot module can be programmed to automatically send an electronic message to the patient reminding them of the importance of these results for their upcoming surgery. If there's no response within a specified time, a second reminder can be sent. If the patient continues to not respond or comply, the system can notify a human operator (like a nurse or administrative staff) to reach out to the patient personally.

A second example is a Lack of Communication with Nurses. If the system notices a lack of communication between a patient and nurses (e.g., messages may be left unanswered), the chat bot module can be programmed to send a check-in message to encourage the patient to respond. If the patient doesn't respond, the system can escalate the issue to the medical staff, who can then attempt to contact the patient through more direct means (like a phone call).

A third example is Not Providing Insurance Information. The chat bot module can be programmed to regularly remind patients to submit their insurance information before the surgery. If the patient fails to provide this information after several reminders, the system can notify the hospital's administrative staff, who can then take over and try to obtain the insurance information directly.

A fourth example is Not Providing Required Documentation. The chat bot module can be programmed to remind patients to submit all necessary documentation. If the documents are not submitted in time, the system can send a stronger reminder emphasizing the importance of these documents. Continued non-compliance can again be escalated to the hospital's administrative staff.

A fifth example is Delay in Responding to Communications. If a patient is consistently slow to respond to messages, the chatbot module can be programmed to send a message encouraging them to be more responsive, reminding them of the importance of timely communication for their upcoming procedure. If slow responses continue, the system can alert a member of the medical team or administrative staff to try to establish more direct communication.

By building a dynamic plan of action around these triggers, the system can proactively intervene when a potential cancellation or no-show is detected, thereby reducing the chances of unexpected cancellations and no-shows. For example, a combination of automated reminders and escalations may be used, and then human intervention may be used as a last resort when the automated measures are not successful. This approach maximizes efficiency while helping the patients receive the care and attention they need.

The user software application (which may be referred to as a user app) can be executed by an electronic device that is in the form of a phone, tablet, or personal computer. In the example embodiment, the user app is software that is located on the user's personal electronic device of choice. Through the user app, the user may contact the clinic or hospital to initiate and manage appointment schedules. These interactions are further managed through the service management server, where each interaction is logged and analyzed as part of the generation of predictive features.

The clinic user interface and database may be software implemented and executed by an electronic device that is in the form of a local device, such as a desktop computer, laptop, notepad, smartphone, servers and the like used by clinics and hospitals. In at least one embodiment, the clinic user interface and database can be implemented using a cloud servers with cloud storage. The system also incorporates known communication software that allows for communication between the patients and the clinic/hospital through their respective software applications.

The service management server in the example embodiment functions as part of a cloud server. In other embodiments, the service management server can be a physical server. The server can host a number of functions including, but not limited to, the smart chat bot module, the predictive features module, and the cancelation engagement module for no show/cancelation triggers generation. These functions work in conjunction with one another to facilitate communication between the user app and the clinic UI/database, through their respective user interface. The interactions are processed and logged, and further analysis is performed to provide behavior based predictions.

The smart chat bot module may be implemented using software instructions that enable text and/or voice based interaction between the clinic and the patients. For example, the smart chat bot module may be implemented by using natural language processing (NLP), to understand the speech from the users in a communication (e.g., conversation), and also use Large Language Models (LLMs) to understand the situation being discussed in the communication which may include using the latest information available online (e.g., medical and general). The LLM may be fine-tuned over time to make sure answers are direct about any predictions that are provided. If there is missing knowledge that is not available online, the information can be added by a system administrator a machine learning model can be trained to provide answers for this missing knowledge in future communications.

The user app provides an input function for the patient to speak to or chat with a member of the medical service provider, clinic, or hospital, in order to inquire about and manage their desired medical procedure. The stakeholders at the medical facility (e.g., hospital, clinic, doctors office, medical service provider, etc.) may be able to communicate with the patients through the smart chat bot, either through real person agents or a predetermined algorithm. In an example embodiment, a smart chat bot can be programed through software to take input from patients, and produce text/voice extraction data that is sent the stakeholders (e.g., people working at the medical facility) to share data from the patient with the stakeholders. Stakeholders can assign agents to assist or intervene with the smart chat bot, should the need arise.

The service management server also hosts predictive features as part of server functions to identify the probability of surgery cancelations. While the smart chat bot processes interaction between patients and stakeholders or agents to obtain data about this interaction, the predictive features may be used to analyze pattern of communication from this interaction to identify pertinent aspects that may indicate a change in a probability of the patient to postpone, reschedule, or cancel one or more appointments or one or more surgical procedures. Based on the patient's interactions, such predictive features can be further enhanced through aggregated data from various patients.

In some embodiments, artificial intelligence or machine learning models may be utilized to assist and enhance the predictive features of the system. For example, the machine learning model may be trained on most common surgery and non-invasive procedure triggers for cancellations such as those described herein for patients in order to predict a probability of a no show for a patient for a given upcoming appointment. The inputs to the machine learning model may be provided by the features extraction model as described herein.

Should the patient initiate a late cancelation or no show incident, the service management server is configured to log the incident and analyze the interactions leading up to the incident. From such incidents, the predictive features module may be used to identify a set of patterns that may be used to effectively predict the probability of a certain patient's cancelation tendency. The no show/cancelation engagement trigger can be used as factors to enhance predictive features on the service management server. Such no show engagement triggers may be included in a data report that is generated and given to the stakeholders so that they may be notified of interactions leading up to the cancelation incidents, such that the clinics/hospitals may better adjust service schedule management to prevent/avoid future cancellations and any wasted resources which may result therefrom.

The service management server can host a library of templates for quickly creating follow up schedules for different types of surgery and ambulatory services. This library can be accessed so that the templates are available to the patients and users through their respective user app/device. This library may include various electronic forms and electronic surveys that may facilitate efficient scheduling, and enable electronic registrations and check-ins, in order to further reduce probability of late cancelations and no shows. In one embodiment, the system may be configured to provide intake form completion by text or voice, such that patients may have abundant options to see the scheduled process through.

In at least one example embodiment, the service management server may have access to a library in order to host educational content about various types of surgeries for patients. This content may be accessible to patients to allow the patients to receive sufficient information on the pertinent procedures, which may help reduce uncertainty leading up to the surgical dates. The stakeholders at the medical facility may identify the content that they deem may be most helpful to the patients, as well as those shown to have improved surgical schedule adherence in the past. The system is configured to allow stakeholders to store ever an increasing number of videos to better suit the growing demands of patients.

The service management server may be configured to provide smart surveys electronically to collect feedback from patients throughout the scheduling process leading up to an appointment to reduce the possibility of surgery or appointment cancelation. Recommendations may be determined by the service management server where the recommendations are the result of monitoring and analysis of the collection of patient interactions collected between the user app and the service management server.

The predictive features module may be implemented via software and may be used to generate smart recommendations for the stakeholders to reduce the possibility of surgery cancelation. These recommendations may be the result of monitoring and analysis of the collection of patient interactions collected between the user app and the service management server described previously.

In at least one embodiment, the predictive features module, which also may be referred to as a feature extraction module, may be implemented as part of a machine learning (ML) system. The feature extraction module may be used for transforming raw data into a format that can be used by a machine learning model. This involves identifying and extracting the characteristics of the data that are most relevant to the task at hand.

For example, in at least one embodiment in the context of your surgery cancellation prediction model, the predictive features module may be configured to perform data collection, feature identification, feature extraction, feature selection and generating output.

During data collection, the predictive features module may be configured to start by collecting raw data. This data might include, but is not limited to, patient demographic information, appointment details, lab results, insurance information, communication logs, any other relevant information, or any combination thereof.

During feature Identification, the predictive features module may be configured to identify the features, or aspects of the data, that are most likely to be relevant for predicting surgery cancellations. These features are based on understanding of the problem and may include features such as, but not limited to, days until the appointment, patient age, the number of previous no-shows, whether all required documentation has been received, the response time to communications, or any combination thereof. Values for these features may be based on the triggers discussed herein, such as missed lab results, lack of communication with nurses, and the like.

During feature extraction, the predictive features module extracts data for the identified features from raw data. This may involve converting the raw data into a format that can be used by a machine learning model. For example, categorical data might be transformed into numerical data through one-hot encoding, and numerical data might be normalized to a standard scale.

During feature selection, the predictive features module may be configured to perform feature selection, which involves identifying the most important features for the prediction task. This may be done to reduce the dimensionality of the data and improve the machine learning model's performance. Techniques for feature selection can include statistical tests, recursive feature elimination, or methods such as LASSO that incorporate feature selection into the machine learning model training process.

During output generation, the predictive features module may be configured to provide a set of selected features for each patient to the machine learning model. These features effectively summarize the relevant information about each patient in a way that the machine learning model can understand and use to make accurate predictions of the patient being a no show for an upcoming medical appointment and/or surgery.

In various embodiments, an integrated software application (also referred to as an integrated app) on the user device can be provided to communicatively connect insurance providers, hospitals, care provider teams, as well as patients. Depending on the level of service desired, the integrated app can facilitate multifaceted interactions between multiple parties. These interactions allow the predictive features module to analyze a growing number of factors, at least some of which are discussed below, that may assist in making predictions on likelihood of no show engagement triggers.

Referring to FIG. 2, the graph illustrates an example embodiment of a process associated with a patient engagement software to reduce no-shows, schedule filling, and patient experience and access improvement. While this embodiment illustrates one or more of the factors that one of the embodiments of the present system may contain, modifications can be made by those with ordinary skill in the art to better suit individual user needs. The process in FIG. 2 includes, from left to right, self-schedule, appointment alert, reminders, delay notice, e-registration, office payment, survey, no-show engage, lab alert, balance alert, mobile/web pay, payment plan, health campaign and recall.

From the beginning, a set of basic functions may be provided by the service management server to allow patients to conduct service management in a routine manner. A patient may be able to self-schedule services through the user app, which may include program code to generate appropriate appointment alerts and reminders to enhance schedule adherence. A stakeholder of the medical facility may set a certain time period during which the patient may continually modify or change their intended schedule, such that rescheduling may not result in unnecessary waste in resource allocation for the stakeholder. This also provides a level of convenience to the patients.

As time approaches the service date (e.g., medical appointment, surgery date, etc.), a set point may be placed in the timeline to enable a delay notice. This may be a time point where the patient is discouraged from making further adjustments to the surgery schedule. This delay notice can be sent as an electronic message to the electronic devices of the patients or may be generated by the user app and displayed in a Graphical User Interface (GUI) on the patients' electronic devices. The delay notice may also be sent the stakeholders as an electronic message or displayed via a GUI in electronic devices of the stakeholders, so that both parties can make preparation for the upcoming events.

After the delay notice period has been passed, the system 100 may be configured to provide a number of functions through the integrated app to help the patients with schedule adherence, as well as enable the stakeholder's facilities to carry out appropriate preparation based on the patient's responses. These functions include electronic registration, office payments, and pre procedure electronic surveys. These can be predefined electronic forms in the library that are accessible through the user app, or they can be specific electronic forms supplied by an electronic device of the stakeholder at appropriate times. These functions are intended to make the process leading up to the procedure as convenient as possible for both the patients and the stakeholders.

Should the patients decide to carry out a late cancelation, or simply not show up to the scheduled event, the system may be configured to provide a no show engage electronic message to the stakeholder and the associate facilities. This is the point (e.g., outcome) which the system will have ideally predicted, such that resource allocation may be re-planned to have endured minimal waste. The predictive features module may be provided by the service management server may be configured to be able to identify the probability of this no show engage event occurring so that the stakeholder may have planned accordingly, prior to the no show engagement. At this point, the occurrence of the no show engagement triggers subsequent functions that may be used to help improve the overall performance.

This is also the point where the stakeholder may provide the patient with an opportunity to return to the beginning of the sequence and initiate another self-schedule process. Depending on the reason for cancelation or success of the predictive features module, the stakeholder may propose a varying degree of allowance in the subsequent scheduling effort for the patient.

Upon the occurrence of the no show engagement event, the clinic or hospital may initiate a series of functions to better allocate the resources. For example, a Lab alert may be generated, which is an electronic message that is sent to a lab/unit/office of the medical facility so that the medical personnel can update reallocate their resources to better prepare the facilities to provide alternative services for other patients. In another example, a balance alert electronic message may be generated to offset the difference between allocated resources and the reschedule attempt, such that the loss can be minimized and recuperated. For example, the integrated app may then be configured to generate a payment alert for the patients for an appropriate amount that they are responsible for the missed appointment or surgery.

In at least one example embodiment, the integrated app may be configured to facilitate a payment plan to assist the patient in making the payment to address the cost associated with the procedure or cancelation. Additionally, the system (e.g., service management server) may be configured to provide a health campaign electronically to educate the patients and allow them to begin another process of self-scheduling of medical procedures.

Finally, in at least one embodiment, the system may be configured to provide the stakeholder (e.g., via the service management server) with the means to recall the procedure entirely, and/or to initiate another process at conclusion of the process. For example, this means may be a two-way patient chat. In such cases, the system may be configured to provide an integrated two way patient chat for a seamless transition from automated to human communication. This enables the stakeholder to communicate with the patients through either personal or programmed interactions, and switch between each based on specific needs of each patient.

In reference to both FIG. 1 and FIG. 2, in at least one embodiment of the present system, a predictive features function may be provided via software that may be used to monitor one or more parts or the entirety of the process, generally designated by the numeral 200, demonstrated in FIG. 2 and analyze each step of the process 200 to better arrive at a probability for the no show engagement step. Ideally, through analysis of collected data in the database by the predictive features function, each actual no show engage event that occurs may have a higher probability assigned prior to the occurrence of the last minute cancelation, such that the resources allocated can be reassigned efficiently with abundant notice. This may decrease the number of resources that may otherwise be unusable in subsequent reallocation. Together, the system may enable patients to have better confidence in the medical service providing facilities, as well as allow clinics and hospitals to better plan service schedules.

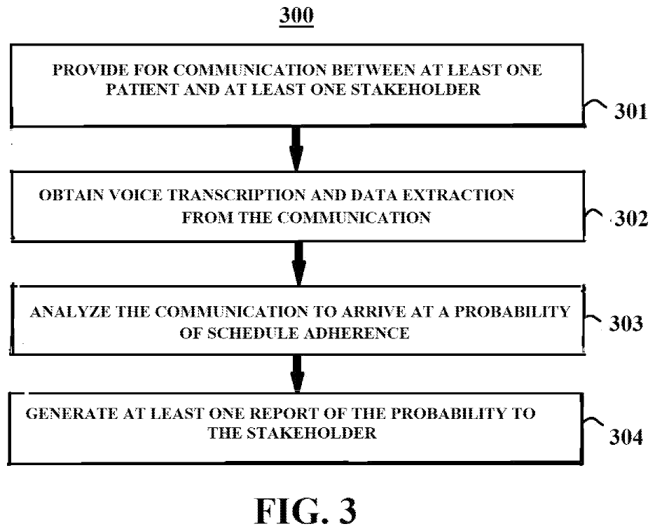

Referring to a FIG. 3, a flowchart of an example embodiment of a method, generally designated by the numeral 300, is shown. This method 300 can be implemented by the system 100 shown in FIG. 1.

At 301, electronic and/or audio communication is provided between at least one patient and at least one stakeholder via the service management server.

At 302, voice transcription and data extraction is performed on the communication. For example, transcription services may be used to transcribe the voice/speech in the communication into text and then extract values from the text.

At 303, the voice transcription and data extraction from the communication is analyzed to arrive at a probability of schedule adherence. This functionality may be provided by the feature extraction module as described previously.

At 304, at least one report or an electronic message of the probability of schedule adherence to the stakeholder is generated.

### Example Cloud Architecture

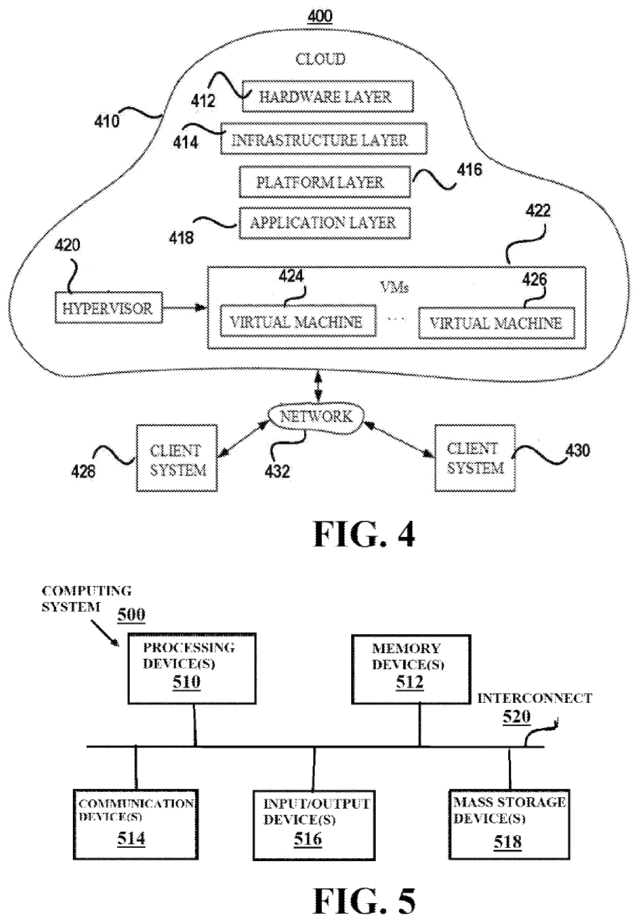

Referring to FIG. 4 with continuing reference to the foregoing figures, an example embodiment of cloud architecture, generally designated by the numeral 400, is shown for implementing the system 100 shown in FIG. 1.

The cloud architecture 400 provides computation, software, data access, and storage services that do not require end-user knowledge of the physical location or configuration of the system that delivers the services. In various embodiments, cloud computing delivers the services over a wide area network, such as the Internet or another electronic communication network, using appropriate communication protocols.

For instance, cloud computing providers deliver applications over a wide area network and they can be accessed through a web browser or any other computing component. Software or components of the cloud architecture 400 as well as the corresponding data, can be stored on servers at a remote location. The computing resources in a cloud computing environment can be consolidated at a remote data center location or they can be dispersed. Cloud computing infrastructures can deliver services through shared data centers, even though they appear as a single point of access for the user. Thus, the components and functions described herein can be provided from a service provider at a remote location using a cloud computing architecture. Alternatively, in at least one embodiment the components and functions can be provided by a conventional server, or they can be installed on client devices directly, or in other ways.

The description is intended to include both public cloud computing and private cloud computing. Cloud computing (both public and private) provides substantially seamless pooling of resources, as well as a reduced need to manage and configure underlying hardware infrastructure.

A public cloud is managed by a vendor and typically supports multiple consumers using the same infrastructure. Also, a public cloud, as opposed to a private cloud, can free up the end users from managing the hardware. A private cloud may be managed by the organization itself and the infrastructure is typically not shared with other organizations. The organization still maintains the hardware to some extent, such as installations and repairs, etc.

As shown in FIG. 4, the cloud architecture 400 includes a cloud 410. The cloud 410 (or each of the different premises on the cloud 410) can include a hardware layer 412, an infrastructure layer 414, a platform layer 416, and an application layer 418.

A hypervisor 420 can illustratively manage or supervise a set of virtual machines 422 that can include a plurality of different, independent, virtual machines 424-426. Each virtual machine can illustratively be an isolated software container that has an operating system and a software application inside it. It is illustratively decoupled from its host server by the hypervisor 420. In addition, the hypervisor 420 can spin up additional virtual machines or close virtual machines, based upon workload or other processing criteria.

A plurality of different client systems 428-430 (which can be end user systems or administrator systems, or both) can illustratively access the cloud 410 over a communication network 432. Depending upon the type of service being used by each of the client systems 428-430, the cloud 410 may provide different levels of service. In one example, the users of the client systems are provided access to application software and databases. The cloud service then manages the infrastructure and platforms that run the application. This can be referred to as software as a service (or SaaS). The software providers operate application software in application layer 412 and end users access the software through the different client systems 428-430.

The cloud provider can also use the platform layer 416 to provide a platform as a service (PaaS). This involves an operating system, a programming language execution environment, database(s) and a web server being provided to the client systems 428-430, as a service, from the cloud provider. Application developers then normally develop and run software applications on that cloud platform and the cloud provider manages the underlying hardware and infrastructure and software layers.

The cloud provider can also use infrastructure layer 414 to provide infrastructure as a service (IaaS). In such a service, physical or virtual machines and other resources are provided by the cloud provider, as a service. These resources are provided, on-demand, by the IaaS cloud provider, from large pools installed in data centers. In order to deploy the applications, the cloud users that use IaaS install operating-system images and application software on the cloud infrastructure 400.

### Example Computer System

Referring now to FIG. 5 with continuing reference to the forgoing figures, shown therein is an example embodiment of a computer system, generally designated by the numeral 500, for use by the system 100 shown in FIG. 1.

The hardware architecture of the computing system 500 that can be used to implement any one or more of the functional components described herein. In some embodiments, one or multiple instances of the computing system 500 can be used to implement the techniques described herein, where multiple such instances can be coupled to each other via one or more networks.

The illustrated computing system 500 includes one or more processing devices 510, one or more memory devices 512, one or more communication devices 514, one or more input/output (I/O) devices 516, and one or more mass storage devices 518, all coupled to each other through an interconnect 520. The interconnect 520 can be or include one or more conductive traces, buses, point-to-point connections, controllers, adapters, and/or other conventional connection devices to facilitate data communication and/or power distribution. Each of the processing devices 510 controls, at least in part, the overall operation of the processing of the computing system 500 and can be or include, for example, one or more general-purpose programmable microprocessors, digital signal processors (DSPs), mobile application processors, microcontrollers, application-specific integrated circuits (ASICs), programmable gate arrays (PGAs), or the like, or a combination of such devices.

Each of the memory devices 512 can be or include one or more physical storage devices, which can be in the form of random access memory (RAM), read-only memory (ROM) (which can be erasable and programmable), flash memory, a miniature hard disk drive, or other suitable type of storage device, or a combination of such devices. Each mass storage device 518 can be or include one or more hard drives, digital versatile disks (DVDs), flash memories, or the like. Each memory device 512 and/or mass storage device 518 can store (individually or collectively) data and program instructions for software, which when executed by the one or more processing devices 510, configure the one or more processing device(s) 510 to execute operations to implement any of the techniques, functions and methods described above in accordance with the teachings herein.

Each communication device 514 can be or include, for example, an Ethernet adapter, cable modem, Wi-Fi adapter, cellular transceiver, baseband processor, Bluetooth or Bluetooth Low Energy (BLE) transceiver, serial communication device, or the like, or a combination thereof. Depending on the specific nature and purpose of the processing devices 510, each I/O device 516 can be or include a device such as a display (which can be a touch screen display), audio speaker, keyboard, mouse or other pointing device, microphone, camera, etc. Note, however, that such I/O devices 516 can be unnecessary if the processing device 510 is embodied solely as a server computer.

In the case of a client device, the communication devices(s) 514 can be or include, for example, a cellular telecommunications transceiver (e.g., 3G, LTE/4G, 5G), Wi-Fi transceiver, baseband processor, Bluetooth or BLE transceiver, or the like, or a combination thereof. In the case of a server, the communication device(s) 514 can be or include, for example, any of the aforementioned types of communication devices, a wired Ethernet adapter, cable modem, DSL modem, or the like, or a combination of such devices.

A software program, app or algorithm, when referred to as “implemented in a computer-readable storage medium,” includes computer-readable instructions stored in a memory device (e.g., memory device(s) 512). A processor (e.g., one of the processing device(s) 510) is “configured to execute a software program” when at least one value associated with the software program is stored in a register that is readable by the processor. In some embodiments, software routines executed to implement the disclosed techniques can be implemented as part of OS software (e.g., MICROSOFT WINDOWS® and LINUX®) or a specific software application, algorithm component, program, object, module, or sequence of instructions referred to as “computer programs.”

Computer programs typically comprise one or more instructions set at various times in various memory devices of a computing device, which, when read and executed by at least one processor (e.g., processing device(s) 510), will cause a computing device to execute functions involving one or more of the disclosed techniques that are in accordance with the teachings herein. In some embodiments, a carrier containing the aforementioned computer program product is provided. The carrier is one of an electronic signal, an optical signal, a radio signal, or a non-transitory computer-readable storage medium (e.g., the memory device(s) 512).

### Example Mobile Device

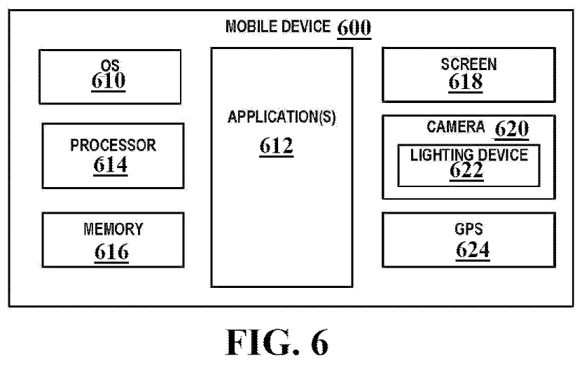

Referring now to FIG. 6 with continuing reference to the foregoing figures, an example embodiment of a mobile device, generally designated by the numeral 600, is shown. The mobile device 600 can be the mobile device shown in FIG. 1.

Mobile device 600 can include operating system 610 and various types of mobile application(s) 612. In some implementations, mobile application(s) 612 can include one or more client application(s) and/or components of a client application that are software programs (e.g., software instructions).

Mobile device 600 can include processor 614 for performing tasks such as signal coding, data processing, input/output processing, power control, and/or other functions, and memory 616 that can be used for storing data and/or code for running operating system 610 and/or mobile application(s) 612. Example data can include web pages, text, images, sound files, video data, or other data to be sent to and/or received from one or more network servers or other devices via one or more wired and/or wireless networks.

Mobile device 600 can include screen 618 and camera 620. The camera 620 can include a lighting device 622. Operating system 610, application(s) 612, processor 614, and/or memory 616 can cooperate to utilize the camera 620 and the lighting device 622 to obtain images.

The mobile device 600 can configure and implement a global positioning system (GPS) 524. The operating system 610 and/or the application(s) 612 can communicate with the GPS 624 to obtain location data.

The detailed description provided above in connection with the appended drawings is intended as a description of examples and is not intended to represent the only forms in which the present examples can be constructed or utilized.

It is to be understood that the configurations and/or approaches described herein are provided as examples, and that the described embodiments, implementations and/or examples are not to be considered in a limiting sense, because numerous variations are possible.

The specific processes or methods described herein can represent one or more of any number of processing strategies. As such, various operations illustrated and/or described can be performed in the sequence illustrated and/or described, in other sequences, in parallel, or omitted. Likewise, the order of the above-described processes can be changed.

Although the subject matter has been described in language specific to structural features and/or methodological acts, it is to be understood that the subject matter defined in the appended claims is not necessarily limited to the specific features or acts described above. Rather, the specific features and acts described above are presented as example forms of implementing the claims.

1. A system to predict and prevent surgical cancelation, the system comprising:
a plurality of user devices for patients and stakeholders to interact with each other through electronic communication, and
a service management server that is in communicatively connected to the plurality of user devices, the service management server comprising a chat module, a predictive features module, and a cancelation engagement module, such that the modules work together to analyze interactions between the user devices to predict a probability of schedule adherence for medical services by the patients.

2. The system of claim 1, wherein the stakeholders include medical service providers, insurance providers, care providers or any combination thereof.

3. The system of claim 1, wherein the user device further comprises of voice transcription and data extraction feature to extract data from communication between a patient and a stakeholder. share data with a plurality of shareholders.

4. The system of claim 1, wherein the service management server further comprises a library of templates to create follow up schedules for various types of surgeries or medical appointments.

5. The system of claim 1, wherein the user devices are further configured to enable the patients to perform electronic registrations and intake form completion.

6. The system of claim 5, wherein the user devices are configured to enable the intake form completion by voice.

7. The system of claim 1, wherein service management server further comprises an education library.

8. The system of claim 1, wherein the user devices are configured to provide at least one smart survey to the patients.

9. The system of claim 1, wherein predictive features module generates smart recommendations for the stakeholders to reduce probability of surgery cancelation.

10. A method to predict surgery schedule cancelation, comprising:
providing for communication between electronic devices of at least one patient and at least one stakeholder,
obtaining voice transcription and data extraction from the communication,
analyzing the voice transcription and data extraction to arrive at a probability of schedule adherence, and
generating at least one report of the probability of schedule adherence to the at least one stakeholder.

11. The method of claim 10, wherein a no show engagement is generated upon cancelation of the at least one patient, and the no show engagement is used to assist in generating at least one report of probability of schedule adherence.

12. A non-transitory computer readable medium storing thereon program instructions, which when executed by at least one processor, configure the at least one processor for performing a method for method for predicting surgery schedule cancelation, wherein the method is defined according to claim 10.
